(19) United States
(12) Patent Application Publication
(10) Pub. No.: US 2003/0039577 A1
(43) Pub. Date: Feb. 27, 2003

(54) Method and apparatus for removing contaminants from a contaminated air stream

(76) Inventors: Jerry Nelson, Warwick, RI (US); Craig Andrews, Cambridge, MA (US)

Correspondence Address: EPSTEIN, EDELL, SHAPIRO, FINNAN & LYTLE, LLC, Suite 400, 1901 Research Boulevard, Rockville, MD, 20850-3164, US

(21) Appl. No.: 10/073,889

(22) Filed: Feb. 14, 2002

Related U.S. Application Data
(63) Continuation of application No. 09/661,897, filed on Sep. 14, 2000, now abandoned.
Application No. 09/661,897 is a continuation of application No. 08/932,101, filed on Sep. 17, 1997, now abandoned.

(51) Int. Cl. A61L 9/00; A62B 7/08

(52) U.S. Cl. 422/4; 422/5; 422/120; 422/121

(57) ABSTRACT

A method and apparatus for removing contaminants from contaminated air is accomplished by exposing an incoming air stream from a surrounding area to ultra-violet (UV) radiation to generate ozone in an ozone chamber of the system. The ozone chamber is configured to reduce air through-flow velocity and to provide time for the ozone to mix with the air and oxidize the contaminants. The air stream subsequently enters a germicidal chamber and is again exposed to UV radiation at a different wavelength to destroy bacteria and any ozone in the air stream thus resulting in sterilized air. The system may include various ozone and germicidal chamber configurations to increase residence time within these chambers. Further, the system may be configured for installation within a wall or ceiling, or for mounting on a ceiling fan motor for use with ceiling fans.

### BACKGROUND OF THE INVENTION

1. Technical Field

The present invention pertains to a method and apparatus for removing contaminants from a contaminated air stream. In particular, the present invention pertains to a method and apparatus for exposing a contaminated air stream to ozone generating and germicidal radiation to remove contaminants from that air stream and produce sterilized air.

2. Discussion of Prior Art

Currently, there are numerous devices known as deodorizing machines utilizing ozone and/or ultraviolet (UV) radiation to sanitize and deodorize air in a treated space (i.e., typically a room). Generally, these devices generate large amounts of ozone gas to attain the ozone concentration level necessary to facilitate deodorizing and sterilizing the air. Since ozone concentration levels required for sterilization are sufficiently high to be dangerous to people and/or animals, the use of these devices is typically limited to odors whose removal is difficult (i.e., smoke from fires, organic material spilled on clothing, etc.). Further, when the devices are used in the proximity of people and/or animals, health authorities require that ozone concentrations be reduced to safe levels. However, these reduced or “safe” levels tend to be too low to effectively deodorize and clean the air. Moreover, such devices typically use the germicidal qualities of the ultraviolet radiation to destroy bacteria in the air, but generally either expose the treated space to high levels of radiation, thereby posing health risks to people and/or animals, such as eye trauma and skin lesions, or use very low levels of radiation requiring long exposure times.

The prior art attempts to obviate the aforementioned problems by exposing air from the treated space to ozone and/or UV radiation internally of a device to thereby shield against the above-mentioned harmful effects. For example, Burt (U.S. Pat. No. 3,486,308) discloses an air treatment device having a UV radiation source to sterilize air and a plurality of baffles disposed within the interior of the device housing. The baffles increase an air flow path within the device beyond the dimensions of the device housing to expose the air to radiation for greater periods of time. The UV source produces radiation at a particular intensity to avoid production of ozone.

Japanese Publication JP 1-224030 discloses an air cleaner including an ozone generating section, on ozone-air mixing section and a filter section. The filter section may include a pair of filters having an alkaline component and ozone-purifying material, respectively. Alternatively, the filter section may include a single filter having both an alkaline component and ozone-purifying material to clean air. The air cleaner further includes a winding air flow path for the air stream to traverse during cleaning.

The prior art devices disclosed in the Burt patent and Japanese Publication suffer from several disadvantages. In particular, the Burt device does not utilize ozone, thereby typically only removing bacterial contaminants (e.g., germs) within an air stream and enabling non-bacterial or other contaminants, such as odor causing contaminants, to be returned to a surrounding environment. Conversely, the air cleaner disclosed in the Japanese Publication employs only ozone to clean the air, thereby possibly destroying only a portion of bacterial contaminants within the air stream while returning residual bacterial contaminants to a surrounding environment.

The prior art attempted to overcome the above mentioned disadvantages by employing ozone in combination with UV radiation to remove virtually all contaminants from an air stream. In particular, Chesney (U.S. Pat. No. 2,150,263) discloses a system for internally cleaning, sterilizing and conditioning air within the system. A stream of air is washed and subsequently exposed to UV radiation which generates ozone such that the combination of UV radiation and ozone destroys virtually all bacteria in the air stream. Excess ozone is removed via pumps and utilized for various purposes. Further, Hirai (U.S. Pat. No. 5,015,442) discloses an air sterilizing and deodorizing system wherein UV radiation generates ozone to oxidize and decompose odor-causing components in the air. The ozone is then removed by a catalyzer in conjunction with, and prior to, germicidal UV radiation where the UV radiation also removes germs and sterilizes the air.

The Chesney and Hirai systems suffer from several disadvantages. Specifically, the Chesney system utilizes a single wavelength of UV radiation (e.g., approximately 254 nanometers) which may not be optimal for both generating ozone and destroying bacteria. In fact, this wavelength is generally utilized for its germicidal effects and tends to destroy ozone, thereby degrading the effect of ozone within the air stream. Further, the Chesney system includes a relatively lengthy compartment for treating air, thereby increasing the size and cost of the system. The Hirai system typically utilizes independent radiation sources to generate ozone and germicidal radiation, thereby increasing system cost and complexity. Moreover, the Hirai system does not provide a safety feature where the ozone generating source may be operable when the germicidal or ozone removing source becomes inoperable, thereby leading to emissions of dangerous ozone concentrations from the system. In addition, the Hirai system employs a relatively short, narrow area for ozone generation, thereby degrading the effects of the ozone since there is generally a minimal amount of time and/or space for the ozone to interact with the air.

### OBJECTS AND SUMMARY OF THE INVENTION

Accordingly, it is an object of the present invention to remove contaminants from air within a treated space without emitting ozone or ultraviolet radiation into that treated space endangering people and/or animals.

It is another object of the present invention to reduce costs and minimize the size of an ozone generating chamber within a system for removing contaminants from a contaminated air stream by utilizing an ozone chamber configured to reduce air through-flow velocity (i.e., increase the amount of time air resides within the ozone chamber to reduce air flow velocity through the ozone chamber) to enable ozone generated in the ozone chamber to interact and mix with an air stream.

Yet another object of the present invention is to maintain ozone concentration levels at low or “safe” levels in a system for removing contaminants from a contaminated air stream by utilizing a single radiation source in the system to emit radiation of different wavelengths from different sections of the source to generate ozone and perform germicidal functions on the air stream, respectively. The entire single radiation source can become disabled only as a unit, thereby preventing generation of ozone when the germicidal radiation or ozone-removing section is inoperable.

Still another object of the present invention is to remove contaminants from a contaminated air stream via a system having a bulb holder to facilitate removal and placement of a UV radiation emitting bulb within the system interior.

A further object of the present invention is to control ozone concentration within an ozone generating chamber of a system for removing contaminants from a contaminated air stream by employing a vortex chamber within the ozone generating chamber to control air flow through the ozone generating chamber.

Yet another object of the present invention is to remove contaminants from air within a treated space via a system configured for installation within a wall or ceiling.

Still another object of the present invention is to remove contaminants from air within a treated space via a system configured for installation on a ceiling fan such that ceiling fan motion circulates air through the system.

The aforesaid objects are achieved individually and in combination, and it is not intended that the present invention be construed as requiring two or more of the objects to be combined unless expressly required by the claims attached hereto.

According to the present invention, a method and apparatus for removing contaminants from a contaminated air stream is accomplished by a system in which air is drawn in as a stream into the system housing toward its base and flows through an ozone generating chamber. An ozone generating ultraviolet (UV) radiation source disposed within the ozone chamber emits ultraviolet radiation having a wavelength of approximately 185 nanometers to irradiate the air and generate ozone which oxidizes contaminants (i.e., bacteria, virus, odor-causing element, etc.) residing in the air stream. The ozone chamber is typically configured to include winding or other types of air flow paths, or to induce a vortical air flow to reduce air through-flow velocity and maintain the air stream within the ozone chamber for a residence time sufficient for the ozone to interact with the air. Subsequent to traversing the ozone chamber, the air stream enters a germicidal chamber disposed adjacent the ozone chamber. The germicidal chamber may also be configured to have winding or other types of air flow paths, and includes a germicidal UV radiation source. The germicidal UV radiation source irradiates the air stream and destroys bacteria and breaks down ozone residing therein. The germicidal UV radiation source generates radiation having a wavelength of approximately 254 nanometers to destroy bacteria, viruses, mold spores and ozone remaining after the interaction of air and ozone in the ozone chamber. The radiation source typically includes a single combination UV radiation emitting bulb with different sections of the bulb emitting radiation of different respective wavelengths. The different sections of the bulb are disposed in the corresponding ozone and germicidal chambers. Alternatively, the radiation sources may all be implemented by separate independent bulbs emitting radiation having wavelengths of approximately 185 or 254 nanometers depending upon the chamber in which the bulb is disposed. The bulbs may be powered by a conventional AC ballast (for use in stationary areas), or a conventional DC ballast connected to a battery to enable the system to be portable and used in mobile environments (e.g., cars, boats, trucks, trailers, etc.).

The resulting sterilized air from the germicidal chamber may pass through a catalytic converter disposed adjacent the germicidal chamber to remove any remaining ozone by either converting the ozone back to oxygen, or filtering the ozone from the air stream. An internal fan disposed adjacent the ozone chamber draws air into the system from the base and through the chambers. The system is typically constructed of injection molded plastic wherein the system housing includes two symmetrical halves. Symmetrical portions of the ozone and germicidal chamber configurations are molded into the respective symmetrical halves such that the symmetrical halves are connected (e.g., snapped together) to form the system. In addition, the system may include a bulb holder that is disposed on the system top surface and extends into the system interior to secure the bulb. The bulb holder extracts the bulb from the system upon removing the bulb holder from the system top surface.

Alternatively, the system may be configured for installation within a wall or ceiling. Specifically, a ceiling or wall unit has substantially the same configuration described above except that the ceiling unit includes a pair of ozone chambers and a pair of germicidal chambers. The ozone and germicidal chambers within each pair are respectively disposed adjacent each other, and function in parallel in substantially the same manner described above. The ozone and germicidal chambers are each constructed within a block of foam wherein the ozone chambers each include a winding path to reduce air through-flow velocity and enable generated ozone to mix and interact with an air stream. Air is directed by the ozone chambers to a corresponding germicidal chamber to remove bacteria from the air stream as described above. The germicidal chambers are disposed adjacent a corresponding ozone chamber and share a common area formed within the foam block. A combination bulb (i.e., emitting radiation of two different wavelengths as described above) and an additional radiation source emitting germicidal radiation are disposed within each germicidal chamber, while a fan, disposed proximate the germicidal chambers, draws air through the system.

In addition, the system may be utilized in combination with ceiling fans to sterilize air in a treated space. In particular, the system is substantially similar to, and functions in substantially the same manner as, the systems described above except that the ceiling fan system does not include an internal fan, and may be of sufficient size to be mounted on a ceiling fan motor. Ceiling fan motion circulates air through the system ozone and germicidal chambers wherein the air is treated as described above and returned to a surrounding environment.

The above and still further objects, features and advantages of the present invention will become apparent upon consideration of the following detailed description of specific embodiments thereof, particularly when taken in conjunction with the accompanying drawings wherein like reference numerals in the various figures are utilized to designate like components.

### BRIEF DESCRIPTION OF THE DRAWINGS

FIG. 1 is a side view in perspective of a system for removing contaminants from a contaminated air stream including a combination exhaust vent and bulb holder to facilitate placement and removal of an ultra-violet (UV) radiation emitting bulb from the system interior according to the present invention.

FIG. 2 is a top view of the combination exhaust vent and bulb holder of the system of FIG. 1.

FIG. 3 is a side view in elevation and partial section of the system of FIG. 1.

FIG. 4 is a side view in elevation and partial section of an alternative configuration for the ozone and germicidal chambers of the system of FIG. 1 according to the present invention.

FIG. 5 is a perspective view in partial section of the system of FIG. 4 diagrammatically illustrating the air flow path through that system.

FIG. 6 is a side view in elevation and partial section of yet another configuration for the ozone and germicidal chambers of the system of FIG. 1 according to the present invention.

FIG. 7 is a side view in elevation and partial section of still another configuration for the ozone and germicidal chambers of the system of FIG. 1 according to the present invention.

FIG. 8 is a side view in elevation and partial section of a helical configuration for the ozone and germicidal chambers of the system of FIG. 1 according to the present invention.

FIG. 9 is a side perspective view in partial section of a portion of the system of FIG. 1 having a further configuration for the ozone and germicidal chambers according to the present invention.

FIG. 10 is a side perspective view in partial section of the system of FIG. 1 having an ozone chamber configured for selectively producing a vortical or radial air flow through the ozone chamber according to the present invention.

FIG. 11 is a top view in plan of the ozone chamber of FIG. 10 having inlet passages and a valve to control air flow through and within the ozone chamber according to the present invention.

FIG. 12 is a front view in elevation of the valve of FIG. 11.

FIG. 13 is an exploded view in perspective of a system for removing contaminants from a contaminated air stream, typically configured for installation within a ceiling or wall according to the present invention.

FIG. 14 is a view in perspective of a portion of the system of FIG. 13 diagrammatically illustrating the air flow path through the system.

FIG. 15 is an exploded view in perspective of an alternative embodiment of the system of FIG. 13.

FIG. 16 is a view in perspective of a system for removing contaminants from a contaminated air stream, typically configured for installation on a ceiling fan, diagrammatically illustrating air flow entering and being exhausted from the system according to the present invention.

FIG. 17 is a view in perspective of the system of FIG. 16 mounted on a ceiling fan.

### DETAILED DESCRIPTION OF THE PREFERRED EMBODIMENTS

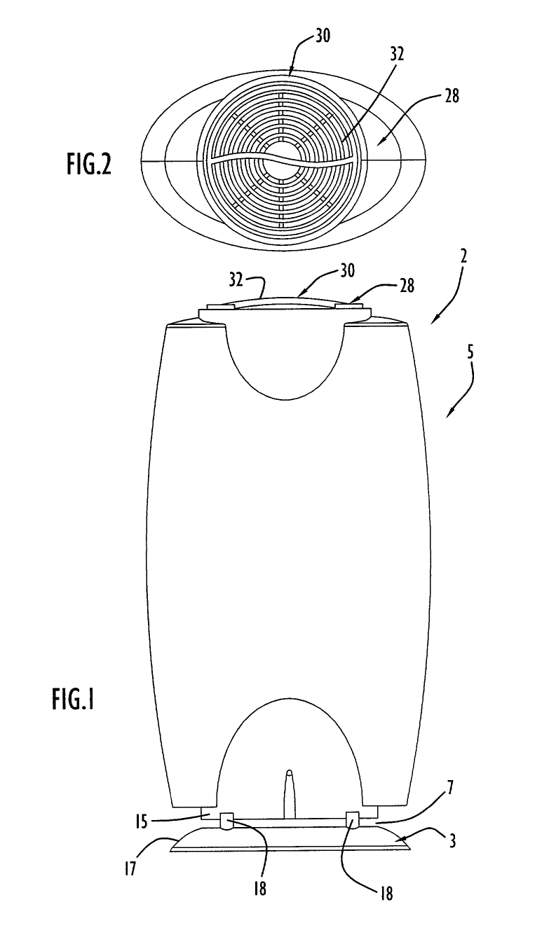

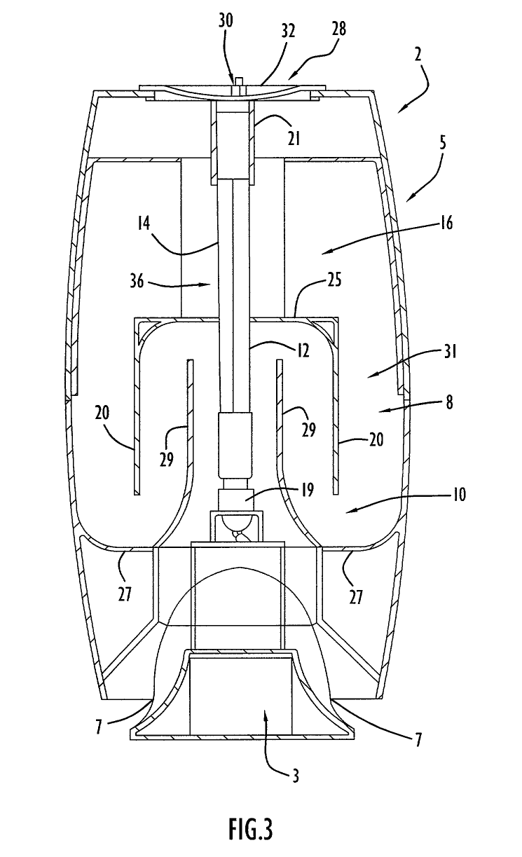

A system for removing contaminants from a contaminated air stream including a combination exhaust vent and bulb holder is illustrated in FIGS. 1-3. Specifically, system 2 includes a generally cylindrical housing 5 extending from a base 3, ozone and germicidal chambers 8, 16, a UV radiation source 36, typically implemented by a combination ultraviolet radiation emitting bulb and disposed at the approximate center of the ozone and germicidal chambers, a ballast (not shown), preferably conventional, for supplying current to radiation source 36, and an internal fan (not shown) for drawing air through the system. The radiation source may be implemented by a single bulb having an ozone section 12 and germicidal section 14 emitting radiation at different wavelengths (i.e., 185 and 254 nanometers) from the ozone and germicidal sections, respectively. Alternatively, the radiation source may be implemented by two independent bulbs disposed in the respective ozone and germicidal chambers. Housing 5 includes a middle portion that has a cross-sectional diameter slightly larger than the cross-sectional diameter of the housing end portions such that the housing has a shape similar to a barrel. Base 3 is typically constructed of an upper and lower support 15, 17 (FIG. 1) wherein the supports are attached to each other via legs or connectors 18 disposed between the supports. Lower support 17 serves as a stand for the system, while upper support 15 typically contains the system electrical components, such as a ballast and fan (not shown) for supplying current to the radiation source and directing air through the system, respectively. However, the fan may be disposed anywhere in the system capable of directing air through the system, while the electrical components may be disposed in the system in any fashion. Legs 18 separate upper and lower supports 15, 17 by a slight distance to form an air intake 7 that serves to permit air to enter the system. Base 3 may alternatively be constructed of a single support configured to enable air to enter the system.

Air from a surrounding environment is drawn into the system through air intake 7 via the internal fan (not shown) and is directed by the internal fan and the housing internal structure to flow into ozone chamber 8 typically disposed above and adjacent the internal fan and air intake. Ozone chamber 8 includes ozone section 12 of radiation source 36 and a path 10 that serves to decrease air through-flow velocity (i.e., the path increases residence time of an air stream within the ozone chamber, thereby decreasing velocity of the air stream through the chamber) and enhance ozone distribution within the air stream. The end of radiation source 36 adjacent ozone section 12 is placed within a power connector 19 disposed at the approximate center of the bottom portion of the ozone chamber. It is to be understood that the terms “top”, “bottom”, “upper”, “lower”, “front”, “rear”, “back”, “side”, “horizontal” and “vertical” are used herein merely to facilitate descriptions of points of reference and do not limit the present invention to any specific configuration or orientation. Power connector 19 provides current from a ballast (not shown) to radiation source 36, and may be implemented by any conventional or other type of connector. The end of radiation source 36 adjacent germicidal section 14 is placed within bulb holder 30 of exhaust vent 28 wherein the exhaust vent is disposed on the system top surface with the bulb holder extending from the exhaust vent into the system interior. The radiation source extends from power connector 19 toward bulb holder 30 with the ozone and germicidal sections disposed at the approximate center of the respective ozone and germicidal chambers. Alternatively, system 2 may be configured such that radiation source 36 has a portion of germicidal section 14 disposed within the ozone chamber to enable the path to combine the effects of ozone producing and germicidal radiation to further remove contaminants from the air stream.

Path 10 receives an air stream entering ozone chamber 8 from the approximate bottom center of the ozone chamber proximate ozone section 12 and transversely directs the air stream away from ozone section 12 toward housing 5. Ozone section 12 generates ozone within the air stream, while path 10 reduces air through-flow velocity and enables the ozone to mix and interact with the air stream to oxidize contaminants. A plurality of reversing passages 31 form path 10 wherein the passages are defined by spaces between a plurality of walls 20, 29. Walls 20, 29 are disposed within the ozone chamber between upper and lower ozone dividers 25, 27 that define the confines of the ozone chamber. Walls 20 each extend from an end of upper divider 25 substantially parallel to each other toward lower divider 27 wherein the length of each wall 20 is slightly less than the distance between the upper and lower dividers to form a gap that enables the air stream to enter and traverse succeeding passages 31. Similarly, walls 29 each extend from an intermediate portion of lower divider 27 such that ozone section 12 is disposed between walls 29 and walls 29 are disposed between walls 20. Walls 29 each extend from lower divider 27 toward upper divider 25 wherein the length of each wall 29 is slightly less than the distance between the upper and lower dividers to form a gap that enables the air stream to enter and traverse succeeding passages 31. The upper and lower ozone dividers maintain the air stream within ozone chamber 8, and isolate the ozone chamber from the remaining portions of the housing. Ozone dividers 25, 27 typically extend across the housing interior to prevent the air stream from bypassing portions of path 10. Lower divider 27 includes an opening toward its intermediate portion to permit the air stream to enter ozone chamber 8, while upper divider 25 is of sufficient size to form gaps between the upper divider periphery and housing 5 to permit air to enter germicidal chamber 16 from the ozone chamber. Housing 5 and its internal structural components may be constructed of injection molded plastic or other material and molded within substantially symmetrical halves of the housing. In other words, symmetrical portions of walls 20, 29, ozone dividers 25, 27 and the remaining structural components of housing 5 (e.g., the germicidal chamber) may be molded into corresponding halves of housing 5 such that when the halves are connected (e.g., the halves may be snapped together or connected utilizing any connection technique), the ozone chamber, path and other housing components are formed.

Upon entering ozone chamber 8 from air intake 7, the air stream traverses path 10 wherein the air through-flow velocity is reduced to enable ozone, generated by ozone section 12, to mix with the air stream to oxidize and remove contaminants within the air stream. Further, when a portion of germicidal section 14 is disposed within the ozone chamber, radiation emitted from the germicidal section enhances removal of contaminants from the air stream. Once the air stream traverses path 10, the air stream leaves the ozone chamber and enters germicidal chamber 16. Germicidal chamber 16 includes germicidal section 14 of radiation source 36 that emits UV radiation to destroy contaminants and ozone within the air stream. Housing 5 may include reflective material within the germicidal chamber to enhance the germicidal effect of radiation emitted from germicidal section 14. The germicidal chamber typically shields a user from any visual UV light, and is isolated from the ozone chamber. The sterilized air from the germicidal chamber is exhausted from the system through exhaust vent 28 to the surrounding environment.

Exhaust vent 28 is substantially elliptical, but may be of any shape, and is disposed at the approximate center of the system top surface. Exhaust vent 28 includes bulb holder 30 having a user gripping portion 32 disposed at the approximate center of the exhaust vent. Gripping portion 32 is typically substantially circular, but may be of any shape. Bulb holder 30 further includes a bulb receptacle 21 that typically extends from the approximate center of gripping portion 32 into the germicidal chamber to engage the end of radiation source 36 adjacent germicidal section 14 as described above. Receptacle 21 may include any type of clamp, brace, bracket, receptacle or other mechanism for engaging the radiation source. Bulb holder 30 facilitates removal and placement of radiation source 36 within the system interior. In particular, removal of radiation source 36 from the system interior is facilitated by extracting bulb holder 30 from the system via gripping portion 32. Since radiation source 36 is attached to the bulb holder, the radiation source is also extracted, thereby disconnecting the radiation source from power connector 19. Thus, the radiation source is disabled prior to removal from the system interior to prevent exposure to direct UV light. Conversely, placement of a UV bulb into the system is facilitated by disposing bulb holder 30, containing a UV bulb, back onto the system, via gripping portion 32, with the bulb extending into power connector 19. The bulb is enabled when the bulb is disposed within power connector 19 and gripping portion 32 is placed on the system top surface, thereby preventing exposure to direct UV light. System 2 may be of any shape or size with the bulb holder disposed on the system in any fashion at any location. The housing and its internal structure may be constructed of any suitable material and, by way of example only, the system may include a height of approximately thirteen inches with the housing being constructed of injection molded plastic.

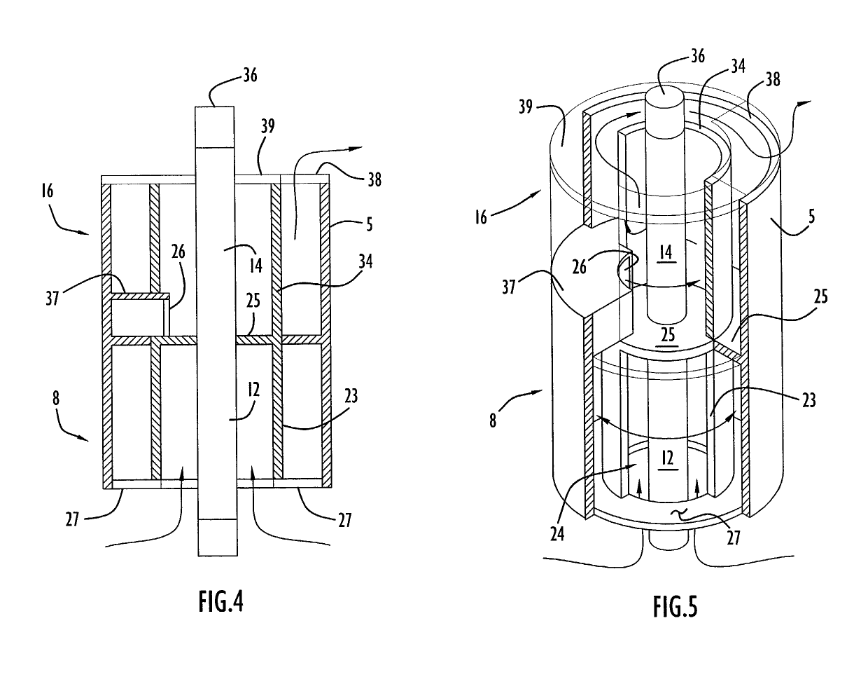

System 2 may include various configurations to reduce air through-flow velocity and enhance distribution of ozone within the air stream as illustrated, by way of example only, in FIGS. 4-5. Specifically, ozone chamber 8 includes substantially annular upper and lower ozone dividers 25,27. The opening within upper divider 25 has dimensions slightly greater than radiation source 36 such that the radiation source is disposed through that opening. Similarly, the opening in lower divider 27 has dimensions greater than the dimensions of the upper divider opening to enable air, drawn through the system by the internal fan as described above, to enter the ozone chamber through the lower divider opening proximate ozone section 12 of radiation source 36. A substantially cylindrical tube 23 extends between the upper and lower divider openings from the periphery of the lower divider opening to form an air flow passage defined by the space between tube 23 and housing 5. Tube 23 includes a cut-out portion 24 extending between the upper and lower dividers that permits air to enter the ozone chamber. Air flows from cut-out portion 24 through the passage to a germicidal chamber entrance 26 angularly offset from cut-out portion 24 by approximately 180°. Entrance 26 is disposed adjacent upper divider 25 to permit air to enter germicidal chamber 16.

Germicidal chamber 16 includes a substantially cylindrical tube 34 that extends from upper divider 25 coincident tube 23. Upper divider 25 is substantially annular as described above and includes a cut-out portion coincident entrance 26 to permit air to enter the germicidal chamber. An elevated portion or ledge 37 is disposed slightly above upper divider 25 and coincident the upper divider cut-out portion to define entrance 26. Air from ozone chamber 8 is directed by ledge 37 through entrance 26 into the germicidal chamber proximate germicidal section 14 disposed within the interior of tube 34. The air traverses a passage defined by the space between tube 34 and housing 5 to germicidal chamber exit 38 angularly offset from entrance 26 by approximately 180°. A substantially annular upper germicidal chamber divider 39 maintains the air within the passage and includes a slot to form the germicidal chamber exit.

The air flow path through the system of FIG. 4 is diagrammatically illustrated in FIG. 5. Specifically, air, drawn through the system by the internal fan as described above, enters ozone chamber 8 proximate ozone section 12 via cut-out portion 24 and the opening within lower divider 27. The air flows in a passage defined between tube 23 and housing 5 toward entrance 26 disposed at an angular offset of approximately 180° from cut-out portion 24. The air stream may flow toward entrance 26 from cut-out portion 24 in either a clockwise or counter-clockwise direction within the passage. The air is directed by ledge 37 through entrance 26 into germicidal chamber 16 proximate germicidal section 14 disposed within the interior of tube 34. Air flows above ledge 37 toward exit 38 in upper divider 39 in either a clockwise or counter-clockwise direction within a passage defined between tube 34 and housing 5. Air exits the germicidal chamber via exit 39 for return to a surrounding environment.

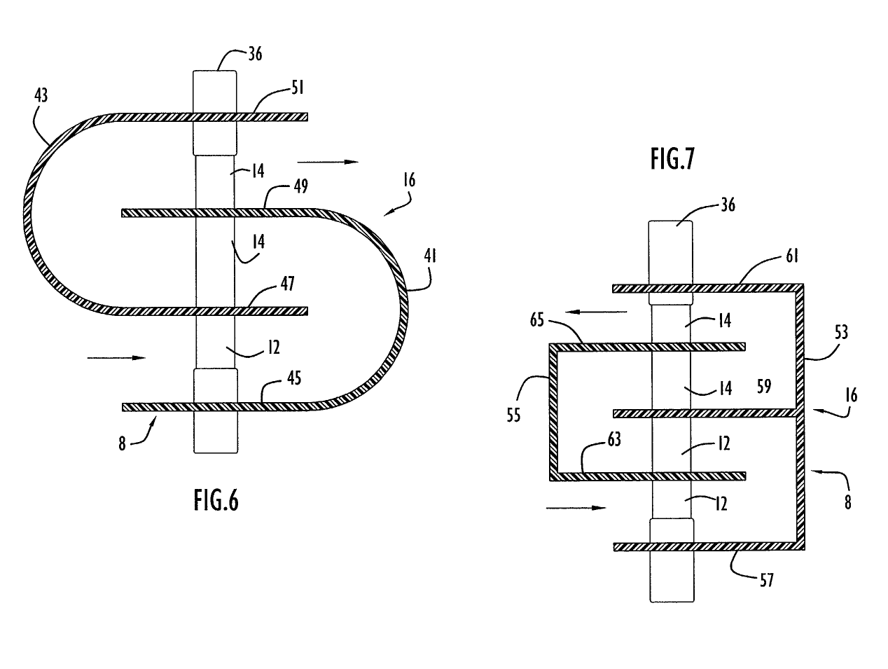

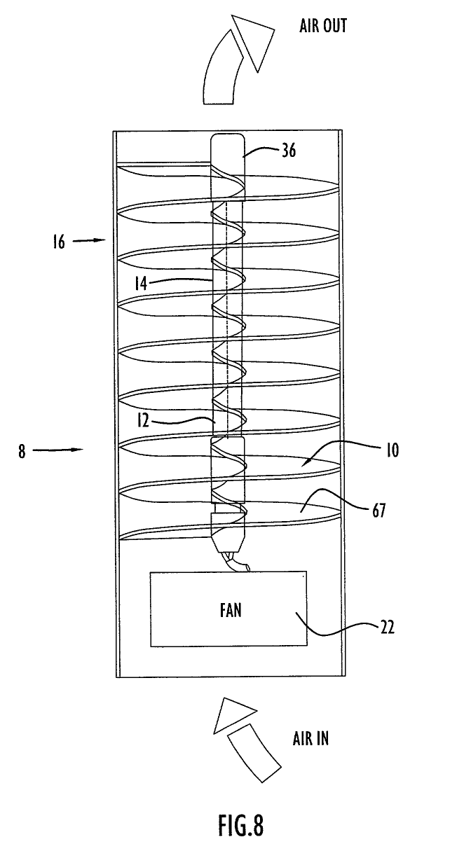

An alternative configuration for the ozone and germicidal chambers is illustrated in FIG. 6. Specifically, the ozone and germicidal chamber configurations may be formed by a pair of ‘U’ shaped walls 41, 43 arranged substantially horizontal with the open portions of the walls in facing relation. Wall 41 includes straight or linear portions 45, 49 connected via a curved portion of wall 41, while wall 43 includes straight or linear portions 47, 51 connected via a curved portion of wall 43. The walls are arranged such that the linear portions 45, 49 of wall 41 are interleaved with the linear portions 47, 51 of wall 43 to form a winding path defined by the space between the interleaved portions and the interior of walls 41, 43. In other words, walls 41, 43 are arranged such that linear portion 47 of wall 43 is disposed at the approximate center between linear portions 45, 49 of wall 41, while linear portion 49 of wall 41 is disposed at the approximate center between linear portions 47, 51 of wall 43. The air flow, drawn through the system by the internal fan as described above, is directed through the winding path (i.e., as shown by the arrows in FIG. 6) to remove contaminants as described above. Walls 41, 43 define the ozone and germicidal chamber configurations wherein radiation source 36 is disposed through linear portions 45, 47, 49, 51 such that ozone section 12 is disposed between interleaved portions 45, 47 defining ozone chamber 8, while germicidal section 14 is disposed between interleaved sections 47, 49 and 49, 51 defining germicidal chamber 16. The winding path reduces air through-flow velocity within the ozone and germicidal chambers to enhance distribution of ozone in the air stream and to enable exposure of the air stream to germicidal radiation for longer periods of time. Yet another configuration for the ozone and germicidal chambers is illustrated in FIG. 7. Specifically, the ozone and germicidal chamber configurations may be formed by a pair of substantially parallel walls 53, 55. Wall 53 has a greater length than wall 55 and includes dividers 57, 59, 61 respectively extending toward wall 55 from each end and an intermediate portion of wall 53. Wall 55 is disposed coincident an intermediate portion of wall 53 and includes dividers 63, 65 respectively extending toward wall 53 from each end of wall 55. Dividers 57, 59, 61, 63, 65 extend sufficient distances from their respective walls such that the dividers from walls 53, 55 are interleaved to form a winding path through the ozone and germicidal chambers. In other words, walls 53, 55 are arranged such that divider 63 is disposed at the approximate center between dividers 57, 59, while divider 65 is disposed at the approximate center between dividers 59, 61. The interleaved dividers form reversing passages defined by the spaces between the interleaved dividers and walls 53, 55. Radiation source 36 is disposed through dividers 57, 59, 61, 63, 65 wherein ozone section 12 is disposed between dividers 57, 59 of wall 53 defining ozone chamber 8, while germicidal section 14 is disposed between dividers 59, 61 defining germicidal chamber 16. Air, drawn through the system by the internal fan as described above, is directed through the winding path (i.e., as shown by the arrows in FIG. 7) of reversing passages to remove contaminants as described above. The winding path reduces air through-flow velocity within the ozone and germicidal chambers to enhance ozone distribution within the air stream and to enable exposure of the air stream to germicidal radiation for longer periods of time.

Still another configuration for the ozone and germicidal chambers is illustrated in FIG. 8. Specifically, the ozone and germicidal chamber configurations may be formed by a helical or spiral structure 67 extending through the ozone and germicidal chambers. Radiation source 36 is disposed through the approximate center of helical structure 67 wherein the structure spirals about ozone section 12 and germicidal section 14 of radiation source 36 within the ozone and germicidal chambers. Ozone section 12 typically occupies approximately one-third of the bulb and is disposed within ozone chamber 8, while germicidal section 14 occupies the remaining approximate two-thirds of the bulb and is disposed within germicidal chamber 16. An air stream is directed by a fan 22, disposed adjacent ozone chamber 8, to traverse a helical path 10 formed by structure 67 through the ozone and germicidal chambers to remove contaminants as described above. Path 10 forces the air stream to spiral about ozone section 12 within ozone chamber 8, thereby reducing air through-flow velocity to enhance ozone distribution within the air stream. The air stream continues traversing the helical path, and enters germicidal chamber 16 to expose the air stream to germicidal radiation. The helical path enables exposure of the air stream to the germicidal radiation for longer periods of time to further remove ozone and contaminants from the air stream.

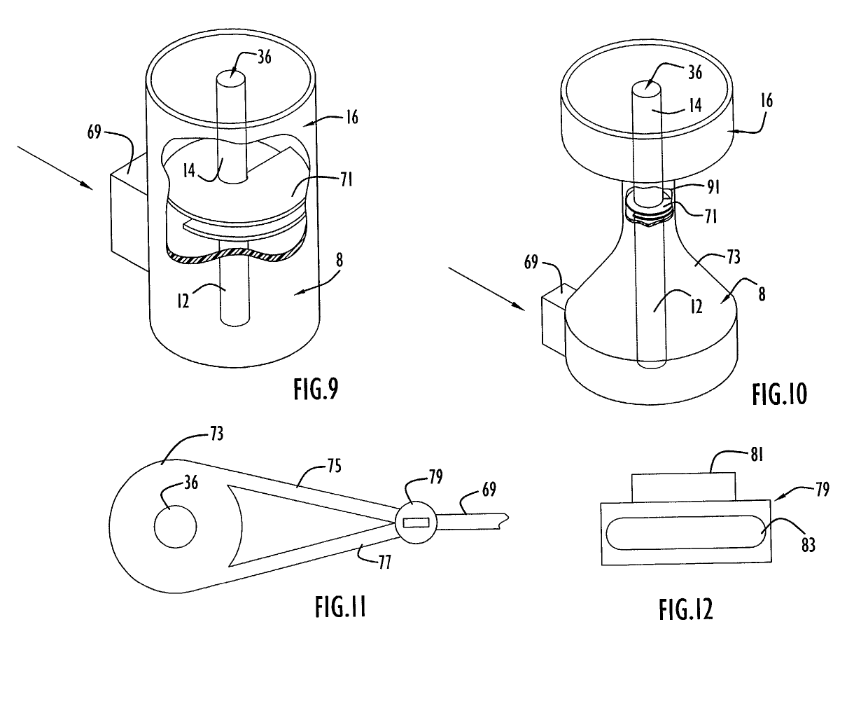

A further configuration for the ozone and germicidal chambers is illustrated in FIG. 9. Specifically, ozone chamber 8 and germicidal chamber 16 include a substantially cylindrical configuration having an inlet 69 disposed proximate ozone chamber 8. The ozone and germicidal chambers each occupy approximately one-half of the substantially cylindrical configuration wherein a helical divider 71 isolates each chamber. Radiation source 36 is disposed through divider 71 such that ozone section 12 resides within ozone chamber 8, while germicidal section 14 is disposed within germicidal chamber 16. Inlet 69 tangentially directs air, drawn through the system by the internal fan as described above, into the ozone chamber such that the air stream flows about ozone section 12 adjacent the ozone chamber walls. Ozone generated by ozone section 12 mixes and interacts with the air to remove contaminants as described above. The air stream flows in this fashion toward helical divider 71 wherein the air stream traverses passages formed in the helical divider to enter germicidal chamber 16. The helical nature of divider 71 enables isolation of the ozone and germicidal chambers, while permitting the air stream to flow in a consistent manner from the ozone chamber into the germicidal chamber. Air flows through the germicidal chamber in a similar fashion to remove bacteria from the air stream as described above.

Alternatively, ozone chamber 8 may be configured to include a vortex chamber 73 to selectively produce a vortical or radial air flow within the ozone chamber as illustrated in FIG. 10. In particular, ozone chamber 8 may include a substantially conical vortex chamber 73 having an air inlet 69 disposed proximate the section of vortex chamber 73 having the greater cross-sectional dimensions. Germicidal chamber 16 is typically substantially cylindrical and disposed adjacent vortex chamber 73 proximate a vortex camber outlet 91 or, in other words, the section of the vortex chamber having the lesser cross-sectional dimensions. A helical divider 71 is disposed between the ozone and germicidal chambers to isolate those chambers. Radiation source 36 is disposed through helical divider 71 such that ozone section 12 is disposed through the approximate center of the vortex chamber, while germicidal section 14 is disposed through the approximate center of the germicidal chamber. Alternatively, radiation source 36 may be implemented by independent sources wherein a substantially annular ozone generating radiation source may be disposed about the periphery of the vortex chamber to generate ozone, while a second radiation source may be disposed in the germicidal chamber to emit germicidal radiation. Air inlet 69 directs the air stream, drawn through the system by the internal fan as described above, into the ozone chamber wherein the air stream is selectively induced to flow tangentially about ozone section 12 along the vortex chamber walls, or radially toward the vortex chamber outlet into the germicidal chamber. A vortical flow reduces air through-flow velocity and enables ozone generated in the ozone chamber to mix and interact with the air stream to oxidize contaminants as described above. A vortical flow is initiated by inlet 69 tangentially directing an air stream into vortex chamber 73. The air stream flows about ozone section 12 along the ozone chamber walls. The tangential air circulation reduces air through-flow velocity and enables generated ozone to mix and interact with the air stream. In essence, the air stream velocity about ozone section 12 increases, while centrifugal force maintains the air stream away from the radiation source. The centrifugal force generally reduces air through-flow through the vortex chamber to maintain the air stream within the ozone chamber. The centrifugal force may become sufficient to prevent virtually all of the air stream from flowing into the germicidal chamber. At lower speeds, the centrifugal force has some effect, but permits the air stream to flow into the germicidal chamber via divider 71. Conversely, when the air stream is divided and the resulting streams are tangentially directed into the vortex chamber in opposing directions, a radial flow is produced, thereby causing air to flow toward the vortex chamber outlet and enter the germicidal chamber with minimal residence time in the ozone chamber.

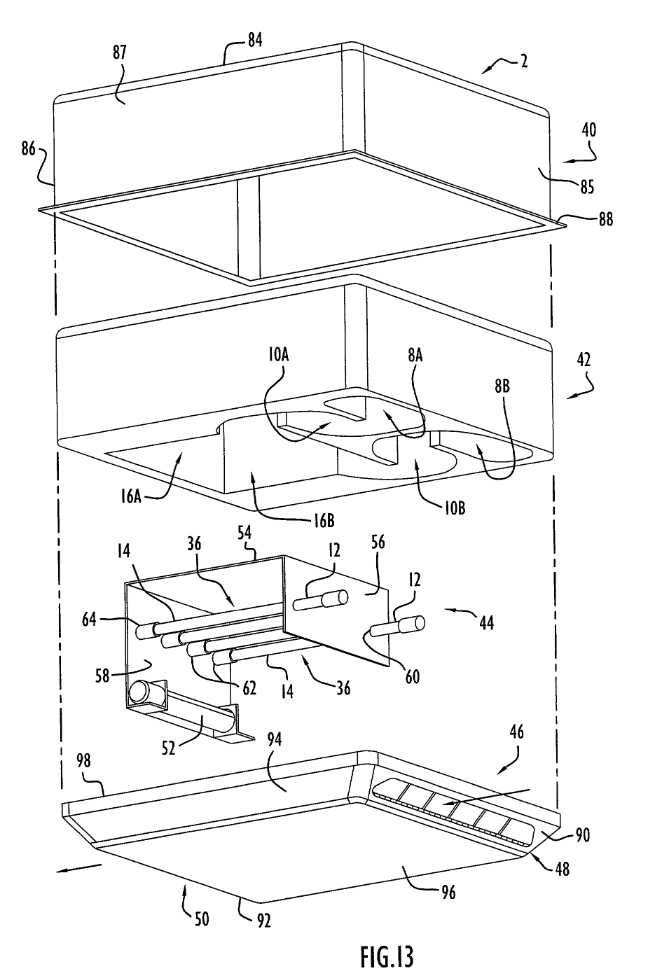

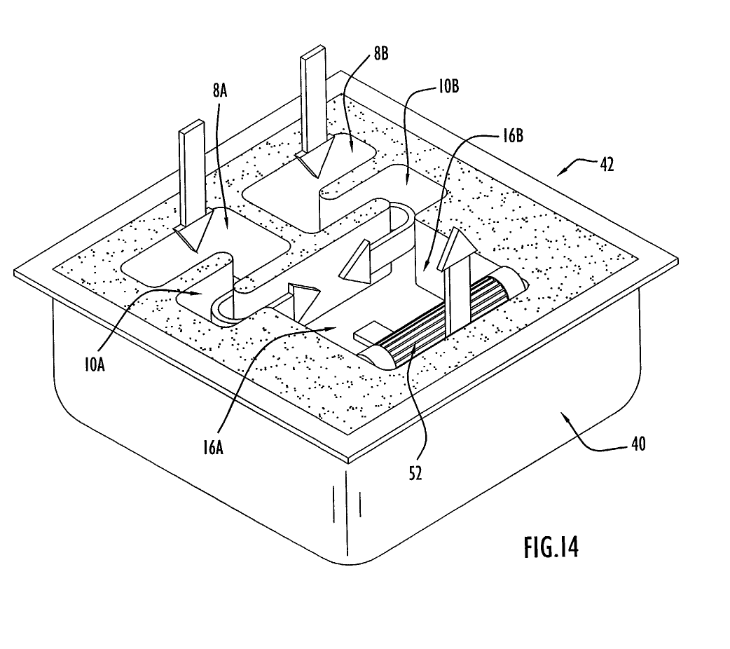

In order to selectively produce a vortical or radial flow within the ozone chamber, the ozone chamber may include a control assembly as illustrated in FIGS. 11-12. In particular, vortex chamber 73 includes inlet passages 75, 77 that tangentially direct air into the vortex chamber in opposing directions (i.e., passage 75 directs air into the vortex chamber in a counter-clockwise direction, while passage 77 directs air into the vortex chamber in a clockwise direction). A valve 79 is disposed at a junction where inlet passages 75, 77 and inlet 69 interface to direct air from inlet 69 through either or both of the passages. The valve is typically in the shape of a disk having a substantially elliptical opening 83 disposed coincident the inlet passages. Another opening (not shown) is disposed on the rear surface of the valve to permit air flow through the valve. A valve actuator 81 is disposed on the valve top surface to control manipulation of the valve and the amount of air flow through each inlet passage. The actuator may be controlled by various mechanical, electrical or other conventional control devices. Air traverses opening 83 to enter inlet passages 75, 77 wherein actuator 81 is manipulated to rotate valve 79 to control placement of opening 83 in relation to the inlet passages to permit air to enter either one or both of the passages. When actuator 81 is manipulated to enable valve 79 to direct air through a single passage, the air enters the vortex chamber and circulates about the radiation source as described above to reduce air through-flow velocity and to enable the generated ozone to mix and interact with the air. When actuator 81 is manipulated to enable valve 79 to direct air through both inlet passages, the opposing air streams enter the vortex chamber and interface to produce a radial flow that reduces residence time within the ozone chamber and causes the air to flow toward the vortex chamber outlet and into the germicidal chamber as described above. Thus, controlling air through-flow velocity or residence time within the ozone chamber enables control of the ozone generated, and hence, the ozone concentration within the air stream. In other words, manipulation of valve 79 via actuator 81 permits certain quantities of air to traverse the inlet passages, thereby controlling the air flow pattern and residence time within the chamber that determines ozone concentration within the air stream. Other mechanisms may be utilized to control air flow in the vortex chamber, such as disposing spiral or other types of walls within the vortex chamber to direct air flow. For further details on the structure, operation and control of flow utilizing vortex chambers and other fluid regulators, reference is made to U.S. Pat. Nos. 3,198,214 (Lorenz) and 4,276,943 (Holmes), the disclosures of which are incorporated herein by reference in their entireties.

It is to be understood that vortex chamber 73 may include any shape or dimensions wherein air may enter the vortex chamber and be directed toward a vortex chamber outlet. For example, in applications requiring compact systems, the ozone and/or vortex chamber may be implemented by a passage having a relatively small depth, while maintaining residence time within the ozone chamber for interaction of ozone with the air stream by producing a vortical flow as described above. In addition, ozone concentration may be controlled by periodically switching between a vortical and radial flow, or permitting the appropriate amounts of air to flow in inlet passages 75, 77 to control residence time within the ozone chamber as described above.

A system for removing contaminants from an air stream, typically for installation within a ceiling or wall, is illustrated in FIG. 13. The system is similar to the system of FIG. 1 described above except that the system includes a modified housing and a plurality of radiation sources 36, 62. Specifically, system 2 includes a cover or housing 40, chamber block 42, electrical component assembly 44, and a base 46. Base 46, typically constructed of molded plastic or other suitably sturdy material, includes substantially rectangular front, rear, side and bottom walls 90, 92, 94, 96, respectively, that collectively define a base interior. The bottom wall is substantially flat, while the front, rear and side walls are slightly tilted outward to expand the base interior. The upper portions of the front, rear and side walls are not tilted, but rather, extend in a substantially vertical fashion to form a base periphery 98. An intake vent 48 is disposed on base front wall 90, while an exhaust vent 50 is disposed on base rear wall 92. Base 46 may further include dividing walls (not shown) to prevent contact between the incoming contaminated air from intake vent 48 and the outgoing sterilized air to be exhausted through exhaust vent 50, and to distribute the incoming air stream from intake vent 48 to different ozone chambers as described below. A platform (not shown) is disposed slightly below base periphery 98 to cover and form an air chamber within the base interior. The platform is substantially rectangular and includes dimensions slightly less than the dimensions of periphery 98 to form gaps or openings between the platform and periphery adjacent the intake and exhaust vents. The openings enable incoming air to enter the system from intake vent 48, and enable outgoing air from the system to be exhausted through exhaust vent 50. The system may be inserted within a ceiling or wall such that only base 46 is visible within a room to enable the intake and exhaust vents to respectively receive and exhaust air to the room.

Chamber block 42 is typically a substantially rectangular block having cross-sectional dimensions slightly less than base 46 in order to be disposed on the base platform. Block 42 is typically constructed of expandable polypropelene close cell foam, a lightweight and sound and shock absorption material. However, chamber block 42 may be constructed of any other materials capable of forming ozone and germicidal chambers as described below. Chamber block 42 includes a pair of isolated ozone chambers 8a, 8b and a pair of germicidal chambers 16a, 16b wherein each ozone and germicidal chamber functions in substantially the same manner as the respective ozone and germicidal chambers described above. Specifically, ozone chambers 8a, 8b each include path 10a, 10b formed into the foam block serving to reduce air through-flow velocity and enhance ozone distribution within the air stream as described above. The paths are each essentially defined by a winding groove or channel formed in the chamber block to reduce air through-flow velocity and mix generated ozone with the air stream to remove contaminants as described above. Paths 10a, 10b are each formed toward the front portion of the chamber block and extend toward the rear block portion into respective germicidal chambers 16a, 16b. Paths 10a, 10b tend to be mirror images of each other and direct air streams to enter the respective germicidal chambers.

Germicidal chambers 16a, 16b are formed in chamber block 42 adjacent respective ozone chambers 8a, 8b. The air streams from ozone chamber paths 10a, 10b enter the respective germicidal chambers from opposing sides of the chamber block. The germicidal chambers are collectively defined by a substantially rectangular recess formed in the chamber block wherein the germicidal chambers are typically not isolated, but rather, share a common area. Air streams from the ozone chambers are directed through the respective ozone chamber paths and enter germicidal chambers 16a, 16b or, in other words, the chamber block recess. The ozone and germicidal chambers each include radiation sources wherein the radiation sources are disposed on electrical component assembly 44 for disposal within chamber block 42 as described below. The ozone and germicidal chambers may alternatively include any of the configurations described above to reduce air through-flow velocity and enable generated ozone to mix with the air as described above.

Electrical component assembly 44 is typically constructed of sheet metal or other suitably sturdy material and preferably includes two combination radiation sources 36 described above, two radiation sources 62 emitting germicidal radiation similar to germicidal section 14 of radiation source 36, fan 52 and other electrical components for the system, such as ballasts (not shown). The assembly typically includes a top wall 54, a front wall 56 and a rear wall 58. Each wall is substantially rectangular wherein the front and rear walls respectively extend from the top wall front and rear edges substantially perpendicular to the top wall. Top wall 54 has dimensions slightly less than the dimensions of the recess within chamber block 42 forming the germicidal chambers such that assembly 44 is inserted within that recess. Rear wall 58 extends from top wall 54 for a distance substantially similar to the depth of the chamber block recess such that fan 52 is substantially flush with a recess peripheral edge when assembly 44 is disposed within the recess. Front wall 56 extends from top wall 54 substantially parallel to rear wall 58 for a distance slightly less than the extension of the rear wall. Front wall 56 includes an opening 60 disposed toward the approximate center of each front wall side edge, and a pair of receptacles 64 (not shown on front wall 56 in FIG. 13) disposed between openings 60. Similarly, rear wall 58 includes a receptacle 64 disposed coincident each opening 60 and receptacle 64 disposed on front wall 56. Openings 60 disposed on front wall 56 and their corresponding receptacles 64 disposed on rear wall 58 each receive a combination radiation source 36 such that the ozone section of the radiation source extends through opening 60 and is disposed external of the assembly, while germicidal section 14 remains within the assembly. Similarly, corresponding receptacles 64 disposed on the front and rear walls receive radiation sources 62. Receptacles 64 disposed on rear wall 58 typically include connectors to provide current to the radiation sources from a ballast (not shown). Fan 52 is attached to rear wall 58 below the radiation sources, and is typically implemented by a barrel or other type of fan or blower device to draw air through the system.

Assembly 44 is disposed within the chamber block recess forming the germicidal chambers as described above. Top wall 54 is disposed toward the recess bottom, while rear wall 58 is positioned toward the rear portion of the recess with front wall 56 disposed adjacent the ozone chambers. Ozone sections 12 of combination radiation sources 36 extend through openings 60 in assembly front wall 56 into respective ozone chambers 8a, 8b, via a gap provided in the chamber block between the ozone and germicidal chambers, to provide necessary radiation to generate ozone as described above. A germicidal section 14 of a radiation source 36 and an adjacent radiation source 62 of assembly 44 are disposed within each germicidal chamber. Thus, each germicidal chamber includes a germicidal section of the combination radiation source and an additional radiation source to generate the required germicidal radiation. Since the germicidal chambers share a common area, the radiation sources disposed on assembly 44 combine to remove contaminants and ozone from the air streams received from the respective ozone chambers. Chamber block 42 may be constructed of a light colored or white foam having sufficient reflective properties to reflect radiation from the radiation sources within the ozone and germicidal chambers. The reflective property of the ozone and germicidal chambers increases radiation intensity to enhance the effects of the ozone generating and germicidal radiation described above.

Chamber block 42, having assembly 44 disposed therein as described above, is placed on the base platform wherein cover 40 is placed over the chamber block and attached to the base. Cover 40 is typically constructed of injection molded plastic or other suitably sturdy material, and includes substantially rectangular top, front, rear and side walls 84, 85, 86, 87, respectively, that collectively define the cover interior. The bottom portions of the front, rear and side walls include a ledge 88 transversely extending from the respective walls to enable attachment of the cover to the base. The cover interior includes dimensions slightly larger than chamber block 42 to receive and cover the chamber block as described above. System 2 is typically installed within a ceiling or wall wherein air enters the system via intake 48 and sterilized air is returned to the environment via exhaust vent (i.e., as indicated by the arrows in FIG. 13) as described above.

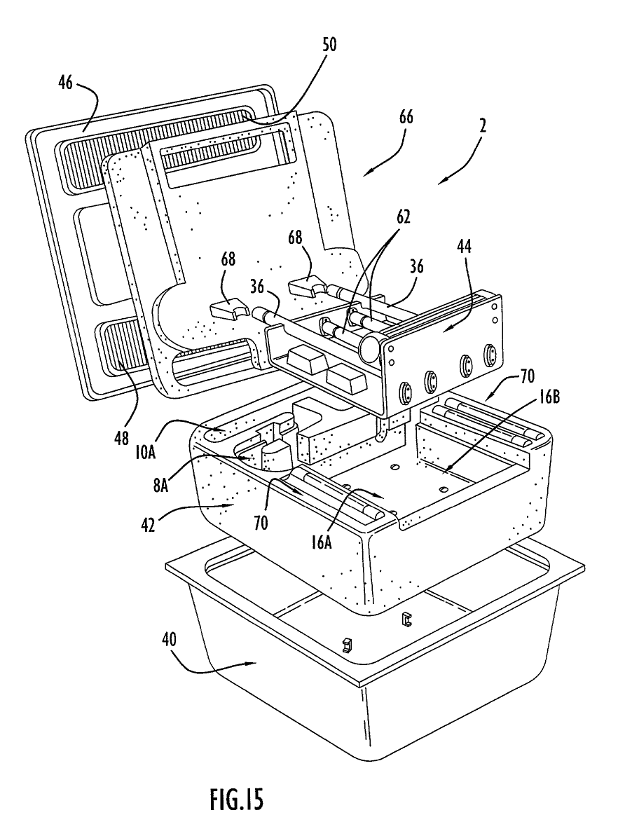

The air flow path through system 2 is substantially similar to the air flow paths through the systems described above and is illustrated in FIG. 14. It is to be understood that FIG. 14 illustrates system 2 in an inverted position relative to FIG. 13 for illustrative purposes and that system 2 is typically mounted in a ceiling or wall in the manner and orientation described above and shown in FIG. 13. Initially, air enters the system via intake vent 48 (FIG. 13) and is divided into two air streams for entry into respective ozone chambers 8a, 8b. The base may include dividers disposed adjacent the intake vent to direct the air stream into the respective ozone chambers. Each air stream enters the respective ozone chamber paths 10a, 10b wherein a corresponding ozone section 12 provides radiation to generate ozone to oxidize and remove contaminants from the respective air streams in substantially the same manner described above. Upon traversing the ozone chamber paths, each air stream enters a corresponding germicidal chamber 16a, 16b. The germicidal chambers are not isolated wherein the air streams from the ozone chambers may interface. The air streams within the germicidal chambers are irradiated by germicidal sections 14 and radiation sources 62 of electrical component assembly 44 (FIG. 13) to remove contaminants and ozone from the air streams in substantially the same manner described above. Air from a surrounding environment is drawn into the system and through the chambers via fan 52 wherein the fan further directs treated air back into base 46 to be exhausted from the system through exhaust vent 50. The system may be of any dimensions, and include any quantity of ozone and germicidal chambers and/or radiation sources. By way of example only, the system typically includes a length of approximately twenty-four inches, a width of approximately twenty-four inches, and an approximate height of eight inches.

Alternatively, system 2 may include a divider 66 to direct air to and from the system as illustrated in FIG. 15. Specifically, system 2 is substantially similar to the system described above for FIG. 13 except that a divider 66 is disposed between base 46 and chamber block 42. The system illustrated in FIG. 15 is inverted relative to the system shown in FIG. 13, however, the system of FIG. 15 is typically mounted in substantially the same manner and at substantially the same orientation described above and shown in FIG. 13. Divider 66 is typically constructed of expandable polypropelene close cell foam or other suitable material, and includes openings that are disposed coincident portions of the ozone and germicidal chambers. The openings permit air from intake vent 48 to enter the ozone chambers and enable air from the germicidal chambers to be exhausted through exhaust vent 50. Divider 66 includes dimensions substantially similar to the cross-section of chamber block 42 and further includes supports or braces 68. The supports are disposed on divider 66 coincident portions of the ozone chambers where ozone sections 12 of the respective radiation sources 36 reside to secure the ozone sections within ozone chambers 8a, 8b when divider 66 is disposed over chamber block 42. The system includes slightly modified ozone chamber paths that provide gaps and/or recesses in the foam for receiving supports 68 and ozone sections 12 of radiation bulbs 36. In addition, system 2 may further include storage compartments 70 disposed on chamber block 42 adjacent germicidal chambers 16a, 16b for storing additional or spare radiation sources. Air is drawn into and is treated by the system in substantially the same manner described above.

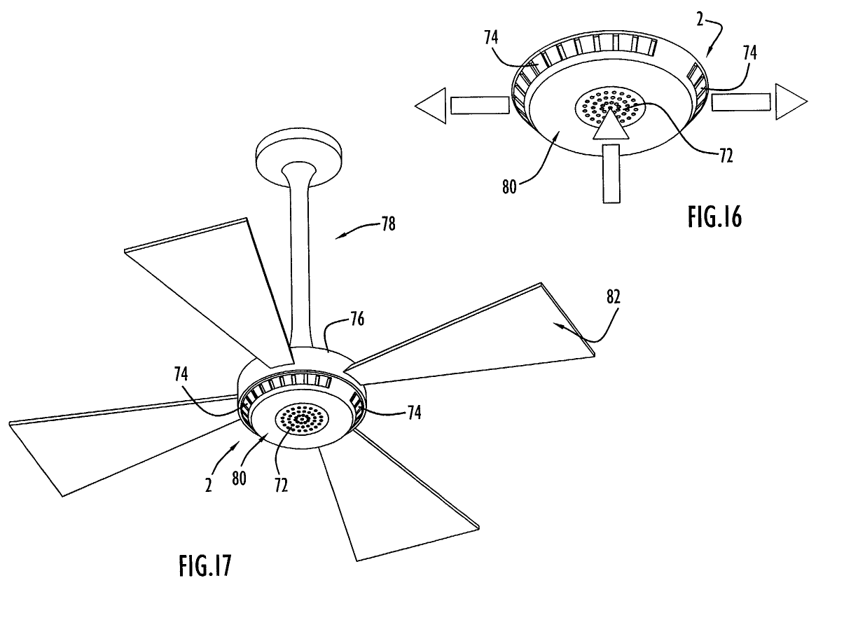

A system for removing contaminants from contaminated air, typically for use in combination with conventional ceiling fans, is illustrated in FIGS. 16-17. Specifically, system 2 typically includes a housing 80, preferably in the shape of a disk, having an intake vent 72 disposed on the housing bottom surface and exhaust vents 74 extending about the housing periphery. The system receives air from intake vent 72 and returns sterilized air to the environment through exhaust vents 74 (i.e., as indicated by the arrows in FIG. 16). System 2 includes dimensions sufficient to mount the system on a bottom surface of a motor housing 76 for a conventional ceiling fan 78. The system generally includes ozone and germicidal chambers having any of the configurations described above, but preferably the vortex chamber configuration, to reduce air through-flow velocity and treat air in substantially the same manner described above. Radiation sources for the system may include the radiation sources described above having appropriate dimensions to accommodate housing 80. Alternatively, the radiation sources may include substantially annular or doughnut shaped combination or single wavelength UV radiation emitting bulbs to accommodate the system housing wherein the ozone and germicidal chambers may be disposed along different and corresponding sections of the combination bulb.

System 2 typically utilizes the air circulation generated by ceiling fan 78 to draw air through the system and, thus, may not necessarily include an internal fan. Specifically, ceiling fan 78 typically circulates air in a room or other space wherein air is drawn up to the fan toward motor housing 76 and is transversely directed away from the fan via the motion of fan blades 82. When system 2 is mounted on motor housing 76 as described above, air drawn to the motor housing is forced into intake vent 72 and through system 2 wherein sterilized air from exhaust vents 74 is transversely directed away from the fan back to the room or space in accordance with the fan generated air circulation. It is to be understood that the systems described above may equally be utilized with ceiling fans wherein the systems are disposed proximate the fans and provide treated air to the air circulation path generated by the fan in substantially the same manner described above.

It will be appreciated that the embodiments described above and illustrated in the drawings represent only a few of the many ways of implementing a method and apparatus for removing contaminants from a contaminated air stream.

The bulb holder system may be of any shape or size, and may be constructed of any suitable materials. The bulb holder system components may be arranged in any manner within the system housing and the base may be implemented by any stand or base capable of supporting that system and its electrical components. The ballasts for the radiation sources may be implemented by any conventional D.C. (e.g., for portable systems) or A.C. ballast or other circuitry to supply current to the radiation sources. The radiation source may be implemented by a single bulb or device capable of emitting radiation at the prescribed wavelengths, or independent sources each emitting radiation at a specified wavelength. The system may include any quantity of radiation sources (e.g., at least one) of any shapes disposed in any manner within the system. The bulb holder may be implemented by any gripping or other device capable of manipulating the bulb. The exhaust vent may be of any shape and may be integral with or independent of the bulb holder (i.e., the bulb holder and vent may be implemented by separate devices). The internal fan may be implemented by any quantity of any conventional or other types of fans or devices for drawing air through the system, such as a fan, blower or device to create a differential pressure in the system to cause air flow through the system. The fan or other devices may be disposed in the system in any manner capable of directing air through the system. Further, the fan or devices may include variable flow rates to cause air to flow through the system at various rates. For example, larger areas may require greater flow rates to enable air within these larger areas to be rapidly and efficiently treated by the system. The system may include any quantity (e.g., at least one) of any shaped ozone and germicidal chambers.

The bulb holder system may be constructed by any quantity of pieces having any portion of the system molded therein wherein the pieces may collectively be attached in any manner to form the system. The bulb connector may be implemented by any conventional or other type of connector. The path may be any path or other configuration capable of reducing air through-flow velocity and enabling the ozone to mix and interact with the air. The ozone chamber may include a portion of the germicidal section of the radiation source to combine the effects of both types of radiation to enhance removal of contaminants. Further, the systems described above may include a catalytic converter or other filter disposed adjacent the germicidal chamber to remove residual ozone from the air stream.

The various ozone and germicidal chamber configurations may be of any size and may be oriented in any fashion, may be implemented by any suitable materials as described above, may utilize any of the radiation sources described above, and may be implemented in any of the systems described above. Further, the radiation source may include any proportion of ozone section to germicidal radiation section wherein the ozone section includes a lesser portion of the source than the germicidal section for the various configurations. Moreover, the combination radiation source only operates when both sections are operable to prevent ozone generation without germicidal radiation to destroy the ozone.

The vortex chamber may be of any shape, preferably forming a loop, and include any dimensions. The vortex chamber may further include any quantity of inlets, valves, tangential or other inlet passages to regulate vertical and radial flow. The valve may be of any shape and may be implemented by any device capable of directing flow into passages. The valve openings may be of any shape and disposed on the valve in any manner capable of regulating air flow. The vortex chamber may include any quantity of radiation sources of any shape (e.g., doughnut shape) to generate the ozone. The germicidal chamber may be of any shape accommodating the vortex chamber.

The ceiling or wall unit may be of any size or shape, or constructed of any suitable material and may include any of the ozone and germicidal chamber configurations described above. The ceiling unit may include any quantity of radiation sources described above disposed in any manner within the chambers. The electrical assembly may be constructed of any suitable material and may support any quantity of electrical components, fans, radiation sources or other components. Further, the electrical and other components may be disposed on the assembly in any fashion. The fan may be implemented by any quantity of any conventional fans or other types of devices described above and disposed anywhere in the system for directing air through the system. The fans or devices may include variable flow rates as described above. The base may be configured to direct air to and from the chambers in any fashion. The ceiling unit components (e.g., block, cover, base, etc.) may be connected or fastened by any conventional or other fastening techniques.

The ceiling fan unit may be of any size or shape and utilize any of the ozone and germicidal chamber configurations or radiations sources described above. The unit may be disposed on the ceiling fan in any manner capable of enabling the ceiling fan to circulate air through the system. Further, any other units may be utilized with the ceiling fan by disposing the units proximate the fan. The ceiling fan unit may be similarly utilized with any fan or blower device capable of circulating air through the system. The ceiling fan unit may be constructed of any suitable materials.

It is to be understood that the present invention is not limited to the specific embodiments discussed herein, but may be implemented in any manner that utilizes ozone generation in combination with a configuration that reduces air through-flow velocity to enable the ozone to interact with the air, and germicidal radiation to remove contaminants from an air stream.

From the foregoing description it will be appreciated that the invention makes available a novel method and apparatus for removing contaminants from a contaminated air stream wherein air is exposed to UV radiation at a first wavelength to generate ozone which oxidizes contaminants in the air while traversing an ozone chamber configured to reduce air through-flow velocity and to enhance ozone distribution in the contaminated air. Subsequently, the air is exposed to UV radiation at a second wavelength to destroy bacteria and ozone in the air.

Having described preferred embodiments of a new and improved method and apparatus for removing contaminants from a contaminated air stream, it is believed that other modifications, variations and changes will be suggested to those skilled in the art in view of the teachings set forth herein. It is therefore to be understood that all such variations, modifications and changes are believed to fall within the scope of the present invention as defined by the appended claims.

What is claimed is:

1. A system for removing contaminants from contaminated air received from a surrounding environment comprising:
an intake to receive said contaminated air from said surrounding environment;
at least one ozone chamber including an ozone radiation source for irradiating said contaminated air to generate ozone to remove contaminants residing in the contaminated air, wherein said at least one ozone chamber is configured to decrease air through-flow velocity and mix the ozone with the flowing air;
at least one germicidal chamber including at least one germicidal radiation source for irradiating the air and ozone mixture to remove residual contaminants and ozone from the mixture resulting in sterilized air;
an exhaust to return the sterilized air back to the surrounding environment; and
air flow control means for controlling the flow of the contaminated air through the system.

2. The system of claim 1 wherein said at least one germicidal chamber includes a first germicidal radiation source, wherein said ozone radiation source and said first germicidal radiation source correspond to ozone and germicidal sections of a single radiation bulb emitting radiation having different wavelengths at different sections of the bulb, wherein a first section of the bulb is disposed in said at least one ozone chamber, and a second section of the bulb is disposed in said at least one germicidal chamber.

3. A system for removing contaminants from contaminated air received from a surrounding environment, comprising:
at least one ozone chamber including an ozone radiation source for irradiating said contaminated air to generate ozone to mix with the air and remove contaminants residing in the contaminated air;
at least one germicidal chamber including a germicidal radiation source for irradiating the air and ozone mixture to remove residual contaminants and ozone from the mixture resulting in sterilized air; and
air flow control means for controlling the flow of the contaminated air through the system;
wherein said ozone and germicidal radiation sources comprise different sections of a single radiation emitting bulb emitting radiation having different wavelengths at different sections of the bulb.

4. In a system for removing contaminants from contaminated air received from a surrounding environment including an air inlet, at least one ozone and germicidal chamber, an exhaust and air flow control means for controlling the flow of the contaminated air through the system, a method of removing contaminants from the contaminated air comprising the steps of:
(a) irradiating said contaminated air in said at least one ozone chamber to generate ozone to remove contaminants residing in said contaminated air;
(b) forming said at least one ozone chamber to decrease air through-flow velocity and mix the ozone with the flowing air to remove the contaminants; and
(c) irradiating the air and ozone mixture in said at least one germicidal chamber to remove residual contaminants and ozone from the mixture resulting in sterilized air.

5. The method of claim 4 wherein:
step (a) further includes:
(a.1) irradiating said contaminated air via radiation having a first wavelength emitted from a first section of a radiation emitting bulb; and
step (c) further includes:
(c.1) irradiating the air and ozone mixture via radiation having a second wavelength emitted from a second section of said bulb;
wherein said first and second wavelengths are different and said first and second sections are disposed in said at least one ozone and germicidal chambers, respectively.

6. In a system for removing contaminants from contaminated air received from a surrounding environment including an air inlet, at least one ozone and germicidal chamber, an exhaust and air flow control means for controlling the flow of the contaminated air through the system, a method of removing contaminants from the contaminated air comprising the steps of:
(a) irradiating said contaminated air in said at least one ozone chamber via an ozone radiation source to generate ozone to mix with, and remove contaminants residing in, said contaminated air;
(b) irradiating the air and ozone mixture in said at least one germicidal chamber via a germicidal radiation source to remove residual contaminants and ozone from the ozone mixture resulting in sterilized air;
wherein said ozone and germicidal radiation sources comprise different sections of a single radiation emitting bulb emitting radiation having different wavelengths at different sections of the bulb.
